(19) United States
(12) Patent Application Publication
(10) Pub. No.: US 2017/0258716 A1
(43) Pub. Date: Sep. 14, 2017

(54) ANTI-CANCER AGENT FOR USE IN COMBINATION THERAPY OF HIGH INTENSITY FOCUSED ULTRASOUND THERAPY AND ANTI-CANCER AGENT THERAPY

(71) Applicant: Tokyo Women's Medical University, Tokyo (JP)

(72) Inventors: Yoshihiro MURAGAKI, Tokyo (JP); Jun OKAMOTO, Tokyo (JP); Masanori MAEDA, Tokyo (JP); Hiroshi ISEKI, Tokyo (JP)

(21) Appl. No.: 15/501,722

(22) PCT Filed: Aug. 3, 2015
(86) PCT No.: PCT/JP2015/071959

§ 371 Date: Feb. 3, 2017

(30) Foreign Application Priority Data
Aug. 4, 2014 (JP) 2014-158945

(51) Int. Cl. A61K 9/107; A61K 31/704; A61M 37/00

(52) U.S. Cl. CPC A61K 9/1075; A61M 37/0092; A61K 31/704; A61M 2037/0007

(57) ABSTRACT

The present invention provides a cancer treatment method in which side effects are inhibited. Specifically, in a combination therapy of a high intensity focused ultrasound therapy at an irradiation intensity of 320 to 700 W/cm² and an anti-cancer agent therapy, an anti-cancer agent containing an anthracycline is used such that the anthracycline in a dose of 0.5 to 7.5 mg/kg body weight is administered to a cancer patient.

### TECHNICAL FIELD

The present invention relates to an anti-cancer agent for use in a combination therapy of a high intensity focused ultrasound therapy and an anti-cancer agent therapy.

### BACKGROUND ART

The high intensity focused ultrasound therapy (hereinafter also referred to as HIFU) is a kind of cancer treatment method. The HIFU is a cancer treatment method of applying pinpoint ultrasound irradiation on a cancer lesion to generate a high temperature (about 90° C.), thereby destroying the cancer lesion.

To enhance a cancer treatment effect, a combination therapy of a HIFU and an anti-cancer agent therapy has been also conducted (Non Patent Literature 1).

### CITATION LIST

### Non Patent Literature

Non Patent Literature 1: “‘Body Friendly’HIFU, which has started to be applied to even a liver cancer treatment” in Cancer Support, [online] Apr. 1, 2013 [searched on July 30, Heisei 26], Internet: http://gansupport.jp/article/cancer/liver/2945.html?print=1>

### SUMMARY OF INVENTION

### Technical Problems

The HIFU using ultrasound has no risk of exposure to radiation, although such exposure to radiation is a concern in a radiation therapy. In addition, the HIFU with pinpoint ultrasound irradiation on a cancer lesion is less invasive in tissue than surgical operation.

However, depending on an irradiation intensity of the ultrasound, a side effect may occur such as a damage in tissue other than a lesion (for example, a skin scald).

In addition, a side effect may also occur depending on a dosage in an anti-cancer agent therapy.

Under these circumstances, there has been a demand for development of a new treatment capable of curing a cancer while inhibiting side effects.

### Solution to Problems

The present inventors have earnestly studied to solve the foregoing problems, and consequently have found that a combination therapy of HIFU and an anti-cancer agent therapy (hereinafter, also simply referred to as “the combination therapy”) is capable of curing a cancer while inhibiting side effects if HIFU at a specific irradiation intensity and an anti-cancer agent therapy using a particular anti-cancer agent in a specific dose are combined. Thus, the present inventors have completed the present invention.

Specially, the present invention relates to the following aspects.
1. An anti-cancer agent comprising an anthracycline, wherein in a combination therapy of a high intensity focused ultrasound therapy at an irradiation intensity of 320 to 700 W/cm² and an anti-cancer agent therapy, the anti-cancer agent as used such that the anthracycline in a dose of 0.5 to 7.5 mg/kg body weight is administered to a cancer patient.
2. The anti-cancer agent according to the above aspect 1, wherein the anthracycline is encapsulated in micellar nanoparticles.
3. The anti-cancer agent according to the above aspect 1 or 2, wherein the anthracycline is epirubicin.
4. The anti-cancer agent according to any one of the above aspects 1 to 3, wherein the cancer patient is a human.
5. The anti-cancer agent according to any one of the above aspects 1 to 4, wherein the anti-cancer agent is administered to the cancer patient before or after the high intensity focused ultrasound therapy.
6. A HIFU treatment apparatus for use in combination with the anti-cancer agent according to the aspect 1, the apparatus being capable of performing ultrasound irradiation at an irradiation intensity of 320 to 700 W/cm², the HIFU treatment apparatus comprising:

a monitor capable of displaying an ultrasound image; and

a safety device which stops the ultrasound irradiation based on the ultrasound image displayed on the monitor.
7. The HIFU treatment apparatus according to the above aspect 6, wherein

in a normal operating state, the monitor displays an ultrasound area representing ultrasound converging at a focal point displayed on the monitor, and soundless areas adjacent to both sides of the ultrasound area, the soundless areas substantially not irradiated with the ultrasound, and

the safety device stops the ultrasound irradiation when the soundless areas are irradiated with ultrasound of 2000 W/cm² or more.

### Advantageous Effects of Invention

As presented in Examples to be described later, the anti-cancer agent of the present invention is capable of curing a cancer while inhibiting side effects when used in a combination therapy of HIFU and an anti-cancer agent therapy. Thus, according to the present invention, it is possible to provide an excellent cancer treatment method less invasive for a patient.

### BRIEF DESCRIPTION OF DRAWINGS

FIG. 1 is a drawing illustrating a schematic configuration of a HIFU treatment apparatus according to an embodiment of the present invention.

FIG. 2 shows an example of a screen displayed on a monitor of an ultrasound monitoring device.

FIG. 3 shows an example of a screen displayed on the monitor of the ultrasound monitoring device.

FIG. 4 is a graph showing results of Examples in the present invention.

### DESCRIPTION OF EMBODIMENT

### Anti-Cancer Agent Containing Anthracycline

An anti-cancer agent of the present invention contains an anthracycline as an active ingredient.

The anthracycline is an antitumor antibiotic substance derived from genus Streptomyces (Streptomyces peucetius). The anthracycline is a substance also called an anthracycline-based anti-cancer agent.

The anthracycline exerts an anti-cancer effect by inhibiting DNA and RNA biosyntheses, inhibiting DNA transcription and copying, damaging DNAs with generation of oxygen radicals, and doing the like.

On the other hand, the anthracycline is also known to cause side effects such as cardiac toxicity and vomitus.

In the present invention, the anthracycline when used in a dose to be described later can exert a treatment effect while inhibiting side effects.

Specific examples of the anthracycline are epirubicin, daunorubicin, doxorubicin, and the like. Among them, the epirubicin is particularly preferable.

The epirubicin is a compound having the following structure.

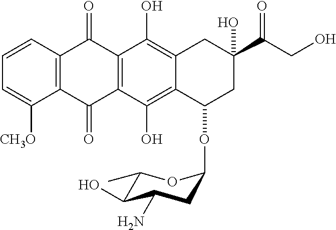

In the present invention, one kind of anthracycline may be used singly, or two or more kinds of anthracyclines may be used in combination.

The anthracycline may be a pharmaceutically acceptable salt (for example, an epirubicin hydrochloride).

The anthracycline may be a natural substance or a synthetic substance.

The anthracycline is a publicly-known substance, and thus can be obtained easily in the market or be prepared.

In the anti-cancer agent of the present invention, the anthracycline is preferably encapsulated in micellar nanoparticles. When the anthracycline is encapsulated in the micellar nanoparticles, the anthracycline can be delivered locally to a cancer lesion with the result that a more powerful anti-cancer effect can be exerted while side effects can be inhibited more.

The average diameter of the micellar nanoparticles is preferably 50 nm to 200 nm, and more preferably 80 to 120 nm. When the average diameter is 50 to 200 nm, the anthracycline can be more localized in the cancer lesion (micellar nanoparticles with the aforementioned diameters tend to be easily accumulated in cancer tissue because the vascular permeability of tumor tissue is remarkably higher than that of normal tissue (EPR effect)).

A method for encapsulating the anthracycline into micellar nanoparticles is already publicly known, and the methods described in International Publication Nos. WO2008/047948 and WO2006/115293 may be used.

In the anti-cancer agent of the present invention, the content of the anthracycline is not particularly limited as long as 0.5 to 7.5 mg/kg body weight of the anthracycline can be administered to cancer patients in a later-described combination therapy of a high intensity focused ultrasound therapy and an anti-cancer agent therapy.

The anti-cancer agent of the present invention may contain a pharmaceutically acceptable carrier in addition to the anthracycline as the active ingredient. The pharmaceutically acceptable carrier is not particularly limited but may be any carrier usable in the anti-cancer agent containing the anthracycline. As the pharmaceutically acceptable carrier, there are, for example, PBS, distilled water, physiological saline, and the like.

As a dosage form of the anti-cancer agent of the present invention, any dosage form applicable to the later-described combination therapy of the present invention can be used without particular limitation. For example, it is possible to select, as needed, any of a liquid agent, an oil agent, an emulsion agent, a soft capsule agent, a hard capsule agent, a tablet agent, a granule agent, a solid agent, and the like.

If the anthracycline encapsulated in micellar nanoparticles is used, the emersion agent is preferable. The anti-cancer agent can be formulated using any of methods known in the pharmaceutical field.

Combination Therapy of High Intensity Focused Ultrasound Therapy and Anti-Cancer agent Therapy

The anti-cancer agent of the present invention is used in a combination therapy of a high intensity focused ultrasound therapy and an anti-cancer agent therapy.

### (1) High Intensity Focused Ultrasound Therapy

The high intensity focused ultrasound therapy (HIFU) is a publicly-known cancer treatment method of performing pinpoint ultrasound irradiation on a cancer lesion to generate a high temperature (about 90° C.), thereby destroying the cancer lesion.

The HIFU using ultrasound has no risk of exposure to radiation, although such exposure to radiation is a concern in radiation therapy. In addition, the HIFU with pinpoint ultrasound irradiation on a cancer lesion is less invasive in tissue than surgical operation.

However, depending on an irradiation intensity of the ultrasound, a side effect may occur such as a damage in tissue other than a lesion (for example, a skin scald).

In the present invention, by conducting ultrasound irradiation at an irradiation intensity to be described later, it is possible to exert a treatment effect while inhibiting side effects.

The irradiation intensity of the ultrasound used in the HIFU of the present invention is 320 to 700 W/cm², preferably 320 to 500 W/cm², and more preferably 350 to 450 W/cm². When the irradiation intensity is 320 to 700 W/cm², the cancer treatment effect can be obtained while side effects are inhibited.

Note that the irradiation intensity of 320 to 700 W/cm² is about ⅕ to about ½ of the irradiation intensity used in the case of a treatment using the HIFU alone. The irradiation intensity of 320 to 700 W/cm² is an irradiation intensity that is, in this technical field, recognized as producing almost no treatment effect even though causing no side effects in the case of the treatment using the HIFU alone.

As irradiation conditions other than the above irradiation intensity, such as a frequency and an irradiation period, for example, the present invention can use any conditions employed in the publicly-known HIFU without particular limitation.

In the HIFU, a trigger irradiation may be performed in which the irradiation intensity of ultrasound is momentarily increased. As conditions of the trigger irradiation, such as a trigger intensity, a trigger period, and a trigger duty ratio, for example, conditions in Examples to be described below may be used.

Here, although the HIFU of the present invention may be carried out by using a publicly-known HIFU treatment apparatus, use of a later-described HIFU treatment apparatus equipped with a safety device makes it possible to further enhance the safely of the treatment.

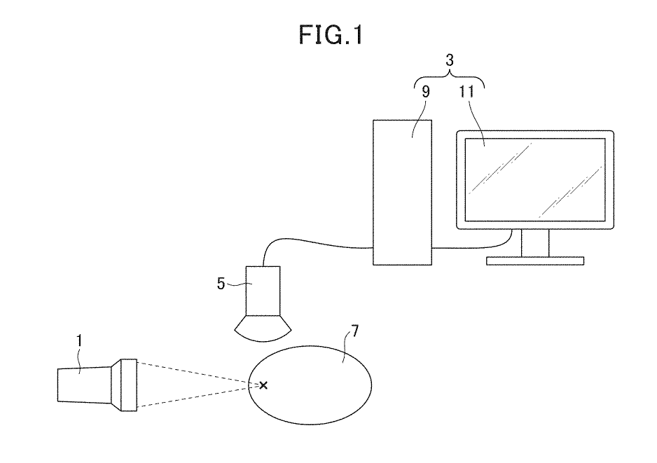

FIG. 1 is a drawing illustrating a schematic configuration of a HIFU treatment apparatus. The HIFU treatment apparatus includes an ultrasound irradiation device 1, such as one used in a high intensity focused ultrasound therapy, which applies convergent ultrasound to a focal point positioned on a treatment location, and an ultrasound monitoring device 3 which monitors the ultrasound applied from the ultra sound irradiation device 1. The ultrasound monitoring device 3 is constituted by devices with which a monitoring target 7 is irradiated with ultrasound applied from a probe 5, reflection of the ultrasound is detected by using the probe 5, and an image of the detection result is formed. The ultrasound monitoring device 3 includes an imaging processing device 9 which forms the image of the detection result of the probe 5, and a monitor 11 which displays the image formed by the imaging processing device 9 in real time

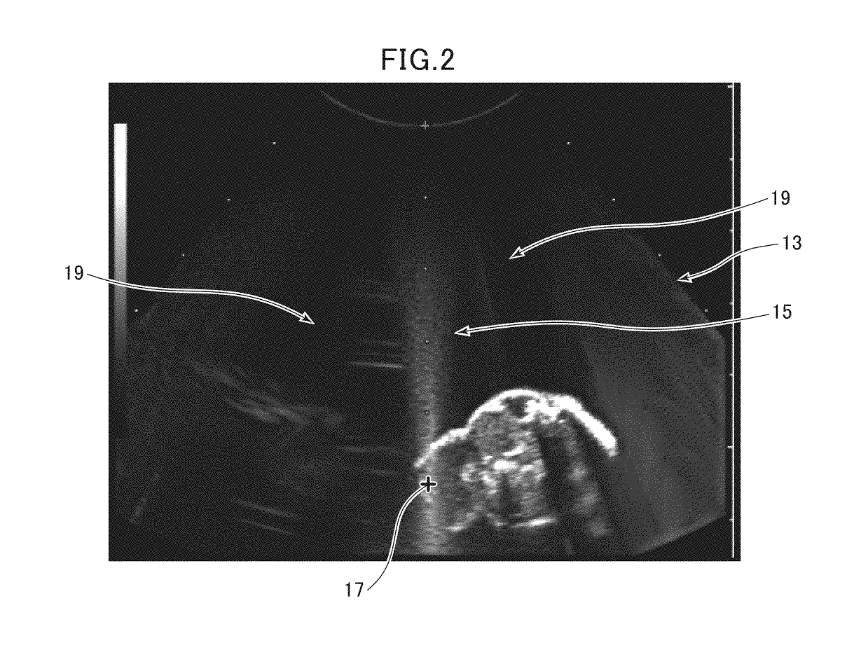

FIG. 2 shows an example of a screen displayed on the monitor 11 of the ultrasound monitoring device 3 while the ultrasound irradiation device 1 is in operation. When the ultrasound irradiation device 1 irradiates a target 7 with ultrasound, the ultrasound applied from the ultrasound irradiation device 1 is visualized and displayed on the monitor 11 as shown in FIG. 2.

FIG. 2 shows the example of the image displayed on the monitor 11. For example, in the case of using a convex-form probe 5, the monitor 11 displays a fan-shaped total area 13 in which an ultrasound area 15 representing the vitalized ultrasound is presented. The ultrasound area 15 is constituted by an infinite number of lines extending in the top-bottom direction of the total area 13, and shows that the ultrasound applied from the ultrasound irradiation device 1 converges at a focal point 17 adjusted to be on an affected part. Then, in the case where the ultrasound irradiation device 1 normally operates and an appropriate power of ultrasound is applied from the probe 5, soundless areas 19 substantially not irradiated with the ultrasound are displayed on both sides of the ultrasound area 15. If the appropriate power of ultrasound is applied from the probe 5, this soundless area 19 has a trapezoidal or triangular shape tapered toward the top side, and the ultrasound is substantially absent or is at 30 W/cm² or less, if any, in the soundless areas 19.

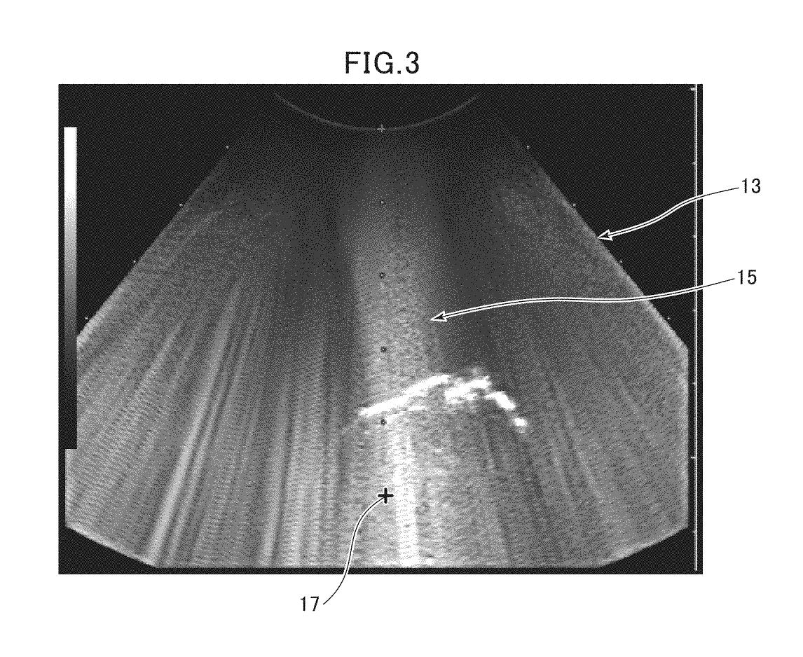

In addition, the ultrasound monitoring device 3 is configured to stop the ultrasound irradiation from the probe 5 when the soundless areas 19 are irradiated with ultrasound of 2000 W/cm² or more. Specifically, when the ultrasound irradiation device 1 operates normally, the ultrasound is not detected actually within the soundless areas 19. However, when the power of the ultrasound applied from the ultrasound irradiation device 1 is increased due to some trouble occurring in the ultrasound irradiation device 1, the ultrasound is detected also within the soundless areas 19 as shown in FIG. 3. For this reason, the ultrasound monitoring device 3 is configured to, when the power of the ultrasound applied from the ultrasound irradiation device 1 is increased due to some trouble occurring in the ultrasound irradiation device 1, detect an increase in the ultrasound within the soundless areas 19 based on an ultrasound image on the monitor 11 and stop the ultrasound irradiation from the ultrasound irradiation device 1.

As a means for detecting an increase in the ultrasound within the soundless areas 19, an image analysis means may be used, for example.

### (2) Anti-Cancer Agent Therapy

The anti-cancer agent therapy in the combination therapy of the present invention is carried out using the aforementioned anti-cancer agent.

In the anti-cancer agent therapy, the anti-cancer agent is administered such that a dose (dosage) of the anthracycline to a cancer patient is 0.5 to 7.5 mg/kg body weight, preferably 1.0 to 5.0 mg/kg body weight, and more preferably 2.0 to 4.0 mg/kg body weight. When the dose is 0.5 to 7.5 mg/kg body weight, it is possible to obtain the cancer treatment effect while inhibiting side effects.

Note that the dose of 0.5 to 7.5 mg/kg body weight is about 1/30 to ⅙ of a dose used in the case of a treatment using an anti-cancer agent therapy alone. Then, the dose of 0.5 to 7.5 mg/kg body weight is a dose that is, in this technical field recognized as producing no treatment effect even though causing no side effects in the case of the treatment using the anti-cancer agent therapy alone.

The number of administrations and an administration interval of the anti-cancer agent may be any number and interval without particular limitation as long as the foregoing dose of the anthracycline can be achieved, and may be set as needed depending on a kind of cancer to be treated, the location of the cancer lesion, conditions of a patient, and the like.

The administration timing of the anti-cancer agent may be before the HIFU, during the HIFU, or after the HIFU. Administering the anti-cancer agent before the HIFU is preferable because the generation of oxygen radicals (oxygen radicals destroy the cancer lesion) from the anthracycline is increased by the ultrasonic irradiation of the HIFU and accordingly a higher treatment effect can be obtained.

Meanwhile, performing the HIFU before the administration of the anti-cancer agent is also preferable because the vascular permeability of the cancer tissue (tumor tissue) (EPR effect) is enhanced by the HIFU, and the anti-cancer agent is more easily accumulated in the cancer tissue and accordingly a higher treatment effect can be obtained.

As an administration route of the anti-cancer agent, any route applicable to the administration of the anti-cancer agent containing the anthracycline can be used without particular limitation. Specific examples of the administration route include a systemic administration (for example, oral administration, intraperitoneal administration, intravenous administration, intra-arterial administration, intramuscular administration, subcutaneous administration, etc.), a topical administration to the cancer lesion, and the like.

The administration route can be set as needed depending on a kind of cancer to be treated, the location of the cancer lesion, conditions of a patient, and the like.

### (3) Indications

Cancers are indications for the present invention. The cancers to be treated are any kinds of cancers to which the HIFU and the anti-cancer agent therapy using the anthracycline are applicable. Specific examples of the cancers include rectal cancer, pancreatic cancer, breast cancer, liver cancer and the like. The present invention is suitably applied to breast cancer and pancreatic cancer among the above cancers.

### (4) Subjects to be Treated

Patients to be treated with the combination therapy of the present invention are all animals suffering from cancers, but are preferably mammals and particularly preferably humans

### EXAMPLES

Next, the effects of the present invention will be specifically described by using Examples, but the present invention is not limited to Examples.

In Examples, using a tumor subcutaneously implanted mouse model, the effects of the high density focused ultrasound therapy (HIFU) in combination with use of epirubicin were examined based on a tumor volume as an index.

### Test Method

Colon-26 (Vendor: RIKEN Bio Resource Center, Registration Number: RCB2 657), a cell line derived from mouse rectal cancer, was subcutaneously transplanted into a ventral part of each 6-week old male CD2F1/Crlj mouse (CHARLES RIVER LABORATORIES JAPAN, INC.) (cell transplanted volume: 1×10⁶ cells/100 μL/animal).

15 days after the transplantation, the mice were divided into 6 groups of 8 animals per group.

Each group received a single intravenous administration (i.v.) (dose: 2.5 mg/kg body weight) of either phosphate buffered saline (PBS) or a drug in which epirubicin micellar nanoparticles (average diameter: 91 nm) internally containing epirubicin were diluted n PBS (epirubicin content: 4.25 mg/mL).

Subsequently, about 24 hours after the administration, a tumor area was exposed to focused ultrasound irradiation (irradiation intensity: 0 (no irradiation), 270 W/cm², or 360 W/cm²). The tumor area was irradiated multiple times (13 times) An irradiation period per irradiation was 30 seconds. The multiple times of irradiations were carried out by shifting the irradiation position immediately after the completion of one time of irradiation, and then performing the neat irradiation (the diameter of the irradiation area per irradiation was 2.5 mm). The detailed irradiation conditions are as follows. Focused ultrasonic irradiation conditions 1 (irradiation intensity of 270 W/cm²)

Frequency: 1.09 MHz

Irradiation intensity: 270 W/cm²

Irradiation period: 30 seconds per irradiation

Trigger intensity: 2 kW/cm²

Trigger period: 20 msec/s

Trigger duty ratio: 2%

Focused ultrasonic irradiation conditions 2 (irradiation intensity of 360 W/cm²)

Frequency: 1.09 KHz

Irradiation intensity: 360 W/cm²

Irradiation period: 30 seconds per irradiation.

Trigger intensity: 2 kW/cm²

Trigger period: 20 msec/s

Trigger duty ratio: 2%

The therapy details of each test group are shown in Table 1.

TABLE 1

| | | | HIFU | | |
|---|---|---|---|---|---|
| | Substance Administration | | Irradiation | Irradiation | Number |
| Test | Substance | Dose (mg/kg | Period and | Intensity | of |
| Group | Administered | body weight) | Times | (W/cm²) | Animals |
| 1 | PBS | — | No | | 8 |
| | | | Irradiation | | |
| 2 | Epirubicin | 2.5 | No | | 8 |
| | | | Irradiation | | |
| 3 | PBS | — | 30 sec, 13 | 270 | 8 |
| | | | times | | |
| 4 | Epirubicin | 2.5 | 30 sec, 13 | 270 | 8 |
| | | | times | | |
| 5 | PBS | — | 30 sec, 13 | 360 | 8 |
| | | | times | | |
| 6 | Epirubicin | 2.5 | 30 sec, 13 | 360 | 8 |
| | | | times | | |

After the ultrasound irradiation, the tumor volumes were measured day by day.

For the measurement of the tumor volume, each animal was anesthetized with isoflurane, then the major axis and minor axis of the tumor were measured by using electronic calipers (Mitutoyo Corporation, #CD67-S15PM), and the tumor volume was calculated using the following equation:

tumor volume (mm³)=major axis (mm)×minor axis (mm)²×0.5.

### Test Results

### (1) Tumor Volume

For each group, the average value and standard error of the tumor volumes on each measurement day at and after the start date of the substance administration were calculated.

Significant difference tests were each carried out between the groups listed below by One-way ANOVA with Dunnett's post tests (between a group and multiple groups) or Student's t-test (between two groups). GraphPad Prism 5 was used for the tests, and the significance level was set to 5% (both sides).

Group 1 vs Group 2

Group 1 vs Groups 3 and 5

Group 2 vs Groups 4 and 6

Group 3 vs Group 4

Group 5 vs Group 6

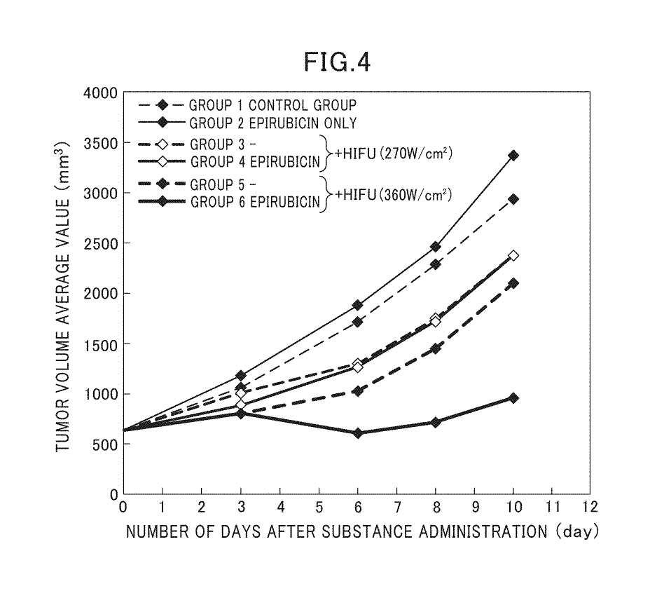

The average value and standard error of each group on each measurement day are shown in Table 2. Table 3 shows statistical analysis (test) results. The results in Table 2 are graphed in FIG. 4.

TABLE 2
Change in Tumor Volume Average Value over Days

| | | | 3 Days After | | 6 Days After | | 8 Days After | | 10 Days After | |
|---|---|---|---|---|---|---|---|---|---|---|
| | Substance | | Substance | | Substance | | Substance | | Substance | |
| | Administration Day | | Administration | | Administration | | Administration | | Administration | |
| | (15 Days After | | (18 Days After | | (21 Days After | | (23 Days After | | (25 Days After | |
| | Transplantation) | | Transplantation) | | Transplantation) | | Transplantation) | | Transplantation) | |
| | Tumor | | Tumor | | Tumor | | Tumor | | Tumor | |
| | Volume | | Volume | | Volume | | Volume | | Volume | |
| Test | Average | Standard | Average | Standard | Average | Standard | Average | Standard | Average | Standard |
| Group | (mm³) | Error | (mm³) | Error | (mm³) | Error | (mm³) | Error | (mm³) | Error |
| 1 | 640 | 92 | 1059 | 168 | 1717 | 301 | 2289 | 394 | 2942 | 464 |
| 2 | 638 | 94 | 1179 | 171 | 1878 | 253 | 2462 | 315 | 3367 | 399 |
| 3 | 640 | 85 | 1006 | 165 | 1298 | 197 | 1748 | 297 | 2379 | 330 |
| 4 | 642 | 86 | 882 | 178 | 1270 | 370 | 1727 | 505 | 2380 | 642 |
| 5 | 639 | 84 | 797 | 125 | 1029 | 134 | 1449 | 196 | 2095 | 305 |
| 6 | 638 | 80 | 808 | 151 | 607 | 86 | 715 | 109 | 955 | 178 |

TABLE 3
Statistical Analysis (Test)

| | 3 Days After | 6 Days After | 8 Days After | 10 Days After |
|---|---|---|---|---|
| Statistical Test | Substance | Substance | Substance | Substance |
| (Tumor Volume) | Administration | Administration | Administration | Administration |
| Unpaired t Test | | | | |
| Group 1 vs Group 2 | ns | ns | ns | ns |
| ANOVA > Dunnett's | | | | |
| Multiple Comparison Test | | | | |
| Group 1 vs Group 5 | ns | ns | ns | ns |
| Group 1 vs Group 3 | ns | ns | ns | ns |
| ANOVA > Dunnett's | | | | |
| Multiple Comparison Test | | | | |
| Group 2 vs Group 6 | ns | ** | ** | *** |
| Group 2 vs Group 4 | ns | ns | ns | ns |
| Unpaired t test | | | | |
| Group 5 vs Group 6 | ns | * | ** | ** |
| Unpaired t Test | | | | |
| Group 3 vs Group 4 | ns | ns | ns | ns |

* p < 0.05, ** p < 0.01, *** p < 0.001

### Group 1 (Control Group)

A tumor growth was observed throughout the observation period. 10 days after the substance administration (25 days after the transplantation), the tumor was proliferated about 4.6 times of the tumor at the start of the administration (15 days after the transplantation).

Group 2 (Epirubicin Administered without HIFU)

Inhibition of the tumor growth as compared to the control group (Group 1) was not observed at all.

Group 3 (No Epirubicin Administered with HIFU (irradiation intensity: 270 W/cm²))

A tendency to inhibit the tumor growth as compared with the control group (Group 1) was observed after the irradiation, but no significant difference was observed.

Group 4 (Epirubicin Administered with HIFU (Irradiation Intensity: 270 W/cm²)

A tendency to inhibit the tumor growth as compared with the control group (Group 1) was observed after the start of the administration. However, no significant difference from Group 2 or Group 3, either, was recognized.

Group 5 (No Epirubicin Administered with HIFU (Irradiation Intensity: 360 W/cm²))

A tendency to inhibit the tumor growth as compared with the control group (Group 1) was observed after the irradiation, but no significant difference was observed.

Group 6 (Epirubicin Administered with HIFU (Irradiation Intensity: 360 W/cm²))

Inhibition of the tumor growth as compared with the control group (Group 1) was observed after the administration and the irradiation, and a tendency of tumor regression was observed 6 days after the substance administration (21 days after the transplantation). Then, significant differences from both Group 2 and Group 5 were recognized.

### (2) Body Weight

Side effects of the combined use of the HIFU and the epirubicin were examined by using the body weight of the mice as an indicator.

The body weight was measured by using a printer-connected electronic balance (Mettler-Toledo international Inc., XP6002SDR). On the day of the substance administration, the body weight was measured before the administration.

A change in the average body weight of Group 6 (Epirubicin administered with HIFU (irradiation intensity: 360 W/cm²)) over time is shown below.

TABLE 4
Change in Average Body Weight (g) of Group 6 Over Time

| | Substance | 1 Day After | 3 Days After | 6 Days After | 8 Days After | 10 Days After |
|---|---|---|---|---|---|---|
| | Administration | Substance | Substance | Substance | Substance | Substance |
| | Day | Administration | Administration | Administration | Administration | Administration |
| | (15 Days After | (16 Days After | (18 Days After | (21 Days After | (23 Days After | (25 Days After |
| | Transplantation) | Transplantation) | Transplantation) | Transplantation) | Transplantation) | Transplantation) |
| Average | 26.1 | 25.7 | 23.2 | 23.4 | 24.0 | 24.1 |
| Body | | | | | | |
| Weight (g) | | | | | | |
| Standard | 0.3 | 0.3 | 0.5 | 0.4 | 0.5 | 0.5 |
| Error (g) | | | | | | |

In Group 6, a decrease in the body weight was observed until 2 days after the ultrasound irradiation (3 days after the substance administration), but thereafter the body weight had a tendency to increase. Since the decrease in the body weight was transient, it was judged that no serious adverse effects occurred due to the combined use of HIFU and epirubicin.

The above test result confirms that the anti-cancer agent of the present invention can cure a cancer while inhibiting side effects in the combination therapy of the HIFU and the anti-cancer agent therapy.

### INDUSTRIAL APPLICABILITY

The anti-cancer agent of the present invention is capable of curing a cancer while inhibiting side effects. Thus, the present invention is applicable in the field of cancer treatment.

1-7. (canceled)

8. An anti-cancer agent comprising an anthracycline, wherein in a combination therapy of a high intensity focused ultrasound therapy at an irradiation intensity of 320 to 700 W/cm² and an anti-cancer agent therapy, the anti-cancer agent is used such that the anthracycline in a dose of 0.5 to 7.5 mg/kg body weight is administered to a cancer patient.

9. The anti-cancer agent according to claim 8, wherein the irradiation intensity is 320 to 360 W/cm².

10. The anti-cancer agent according to claim 8, wherein the anthracycline is encapsulated in micellar nanoparticles.

11. The anti-cancer agent according to claim 8, wherein the anthracycline is epirubicin, daunorubicin or doxorubicin.

12. The anti-cancer agent according to claim 8, wherein the anthracycline is epirubicin.

13. The anti-cancer agent according to claim 8, wherein the cancer patient is a human.

14. The anti-cancer agent according to claim 8, wherein the anti-cancer agent is administered to the cancer patient before or after the high intensity focused ultrasound therapy.

15. A combination therapy for a cancer patient, comprising the step of administering to the cancer patient an anthracycline in a dose of 0.5 to 7.5 mg/kg body weight, before, during or after a high intensity focused ultrasound therapy at an irradiation intensity of 320 to 700 W/cm².

16. The combination therapy according to claim 15, wherein the irradiation intensity is 320 to 360 W/cm².

17. The combination therapy according to claim 15, wherein the anthracycline is encapsulated in micellar nanoparticles.

18. The combination therapy according to claim 15, wherein the anthracycline is epirubicin, daunorubicin or doxorubicin.

19. The combination therapy according to claim 15, wherein the anthracycline is epirubicin.

20. The combination therapy according to claim 15, wherein the cancer patient is a human.

21. The combination therapy according to claim 15, wherein the anthracycline is administered to the cancer patient before or after the high intensity focused ultrasound therapy.

22. A HIFU treatment apparatus for use in combination with the anti-cancer agent according to claim 8, the apparatus being capable of performing ultrasound irradiation with an irradiation intensity of 320 to 700 W/cm², the apparatus comprising:
a monitor capable of displaying an ultrasound image; and
a safety device which stops the ultrasound irradiation based on the ultrasound image displayed on the monitor.

23. The HIFU treatment apparatus according to claim 15, wherein the irradiation intensity is 320 to 360 W/cm².

24. The HIFU treatment apparatus according to claim 15, wherein
in a normal operating state, the monitor displays an ultrasound area representing ultrasound converging at a focal point displayed on the monitor, and soundless areas adjacent to both sides of the ultrasound area, the soundless areas substantially not irradiated with the ultrasound, and
the safety device stops the ultrasound irradiation when the soundless areas are irradiated with ultrasound of 2000 W/cm² or more.

25. A HIFU treatment apparatus for use in the combination therapy according to claim 15, the apparatus being capable of performing ultrasound irradiation with an irradiation intensity of 320 to 700 W/cm², the apparatus comprising:
a monitor capable of displaying an ultrasound image; and
a safety device which stops the ultrasound irradiation based on the ultrasound image displayed on the monitor.
